(19) United States
(12) Patent Application Publication
(10) Pub. No.: US 2025/0057698 A1
(43) Pub. Date: Feb. 20, 2025

(54) TRANSSCLERAL ILLUMINATION FOR VITREOUS VISUALIZATION

(71) Applicant: Alcon Inc., Fribourg (CH)

(72) Inventor: Steven T. Charles, Memphis, TN (US)

(73) Assignee: Alcon Inc., Fribourg (CH)

(21) Appl. No.: 18/761,775

(22) Filed: Jul. 2, 2024

Related U.S. Application Data
(60) Provisional application No. 63/520,121, filed on Aug. 17, 2023.

(51) Int. Cl. A61F 9/007; A61B 90/30

(52) U.S. Cl. CPC A61F 9/0079; A61B 90/30

(57) ABSTRACT

An ophthalmic assembly includes a frame, flange, and light emitter. The frame supports an optical lens. A first end of the flange is connected to the frame. A second end of the flange rests on a pars plana region of the sclera of a patient's eye. The light emitter is connected to the flange proximate the second end to direct light into a vitreous cavity of the patient's eye. This occurs through the sclera at the pars plana region and uniformly illuminates the vitreous. A transscleral illumination system includes the ophthalmic assembly and a processor in communication with the light emitter. A setting of the light emitter may be controllable via the processor, e.g., in response to a user input signal.

### CROSS-REFERENCE TO RELATED APPLICATIONS

The present application claims the benefit of priority to U.S. Provisional Application No. 63/520,121 filed on Aug. 17, 2023, which is hereby incorporated by reference in its entirety for all purposes.

### INTRODUCTION

The present disclosure relates to hardware and associated techniques for visualizing the vitreous body of a patient's eye during an ophthalmic procedure.

The human eye is filled with a collagen-containing gel referred to as the vitreous body, or simply the vitreous. The vitreous of a healthy eye is transparent, a characteristic that permits light entering the eye through the pupil and lens to pass unobstructed to the retina. Aging, injury, and other changes to eye may cause the vitreous to liquify and thin. Within the vitreous cavity, clumped collagen fiber (“floaters”) can cast shadows on the retina. Thus, floaters can adversely affect vision depending on their size, quantity, and location within the vitreous chamber.

In order to properly identify and ultimately treat floaters, a clinician may illuminate the vitreous chamber and view the vitreous in real-time through a set of optics. However, as floaters are largely phase objects that typically absorb only 1-2% of incident light, floaters are notoriously difficult to distinguish with trans-corneal, trans-pupillary illumination. For instance, problematic glare can result from the scattering and reflection of incident light from the cornea, natural lens, or intraocular lens (IOL), or from vitreous. Furthermore, endo-illumination and epi-illumination employs a relatively narrow beam and non-uniform lighting. Both characteristics are suboptimal when visualizing the vitreous.

### SUMMARY

Disclosed herein is a transscleral illumination system and associated control and implementation methodology for visualizing the vitreous of a patient's eye, e.g., during or in preparation for ophthalmic procedures such as laser vitreolysis or vitrectomy surgery. The solutions described herein may be used during an interactive real-time visualization by a clinician, possibly in conjunction with a slit-lamp, in order to afford the clinician with a true dark field view of the patient's vitreous. Unlike the above-summarized trans-corneal, trans-pupillary method, the present approach uniformly illuminates the vitreous chamber by directing light through the sclera and choroid at the ciliary body or pars plana region of the eye as opposed to, e.g., through the cornea, pupil, and lens/IOL.

Illumination is provided by a set of light-emitting diodes (LEDs) or other suitable light emitters. This occurs specifically at or along the pars plana region as noted above, with precise placement of the light emitters being accomplished by an ophthalmic assembly. As part of the disclosed assembly, a tapered/skirt-like portion or flange is connected to or formed integrally with an annular body. The flange circumscribes the cornea and rest directly on the conjunctiva over the sclera.

In particular, an ophthalmic visualization tool is disclosed herein for visualizing a vitreous body within a patient's eye. A non-limiting exemplary embodiment of the visualization tool includes a frame and a light emitter. The frame may include an annular body configured to support an optical lens for viewing the vitreous body, and a flange that is connected to the annular body. The flange has first and second ends. The first end is connected to the frame. The second end is configured to rest on a scleral surface of the eye proximate a pars plana region thereof. The light emitter in this implementation is connected to the flange proximate the second end, and configured to direct light through the scleral surface and into a vitreous cavity of the eye. This occurs at the pars plana region, and has the benefit of uniformly illuminating the vitreous body, as well as eliminating scattered light and reflections that would otherwise occur if illuminating through the cornea, lens, or IOL as noted above.

The annular body could include a set of tabs configured to engage a perimeter edge of the optical lens, with the optical lens possibly being part of the tool.

The first end of the flange, which may have a frustoconical shape, is connected to the annular body. The second end is configured to rest on the scleral surface.

The light emitter in one or more implementations of the tool may include a plurality of LEDs, which in turn may include one or more blue LEDs. At least one of the LEDs is connected to an optical fiber in accordance with certain aspects of the disclosure. The LEDs could also be arranged in a fiber bundle and connected to a plurality of optical fibers.

Also disclosed herein is a transscleral illumination system for visualizing a vitreous body within a patient's eye. The system may include an ophthalmic visualization tool having a frame configured to support an optical lens, as well as a flange having a first end and a second end. As noted above, the first end is connected to the frame. The second end is configured to rest on a scleral surface of the eye proximate a pars plana region thereof. A light emitter is connected to the flange proximate the second end, and configured to direct light through the scleral surface and into a vitreous cavity of the eye. As with the prior-summarized embodiment of the tool, this occurs at the pars plana region to uniformly illuminate the vitreous body. A processor is in communication with the light emitter. A setting of the light emitter is controllable via the processor or analog electronics in such a construction.

The ophthalmic visualization tool in accordance with another implementation includes an optical lens, a frame, a flange, and a plurality of red, green, and blue (RGB) LEDs. The lens is configured for viewing an anterior, middle, or posterior portion of the vitreous body. The frame has an annular body and a neck portion, with the latter being configured to support a perimeter edge of the lens. The flange in this non-limiting/representative construction of the tool has a frustoconical shape inclusive of a first end connected to the neck portion and a second end configured to rest on a scleral surface of the eye at the pars plana region thereof. The LEDs are arranged in an annular arrangement that is connected to the flange in proximity to the second end. The LEDs in the annular arrangement directs light into a vitreous cavity of the eye through the scleral surface, at the pars plana region, to uniformly illuminate the vitreous body.

The above-described features and advantages and other possible features and advantages of the present disclosure will be apparent from the following detailed description of the best modes for carrying out the disclosure when taken in connection with the accompanying drawings.

### BRIEF DESCRIPTION OF THE DRAWINGS

FIG. 1 is an illustration of a representative ophthalmic procedure during which a clinician visualizes the vitreous body of a patient's eye using a transscleral illumination system in accordance with the present disclosure.

FIG. 2 is a schematic illustration of a portion of the transscleral illumination system of FIG. 1.

FIG. 3 is a cross-sectional side view illustration of an exemplary configuration of a visualization tool for use with the transscleral illumination system of FIGS. 1 and 2.

FIG. 4 is a perspective view illustration of the exemplary visualization tool of FIG. 3.

FIG. 5 illustrates light emitters arranged according to one or more implementations.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

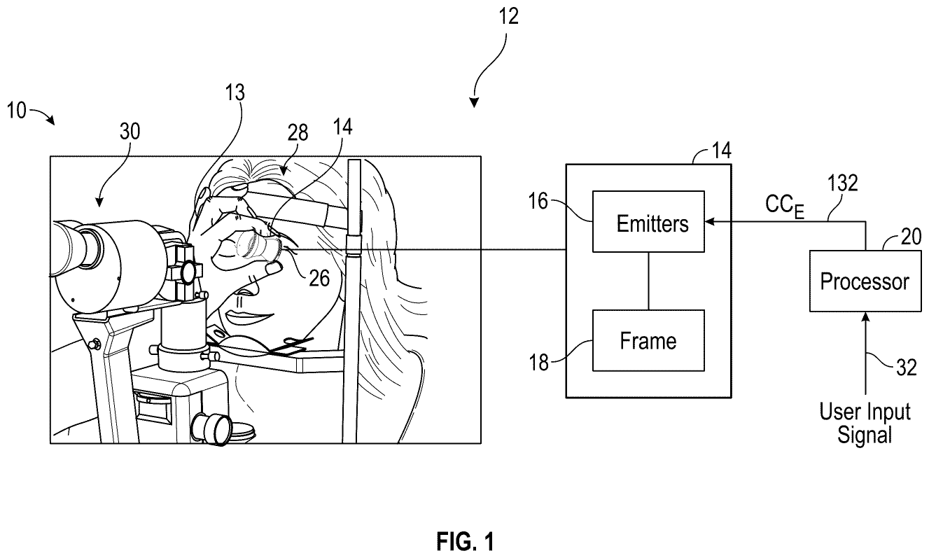

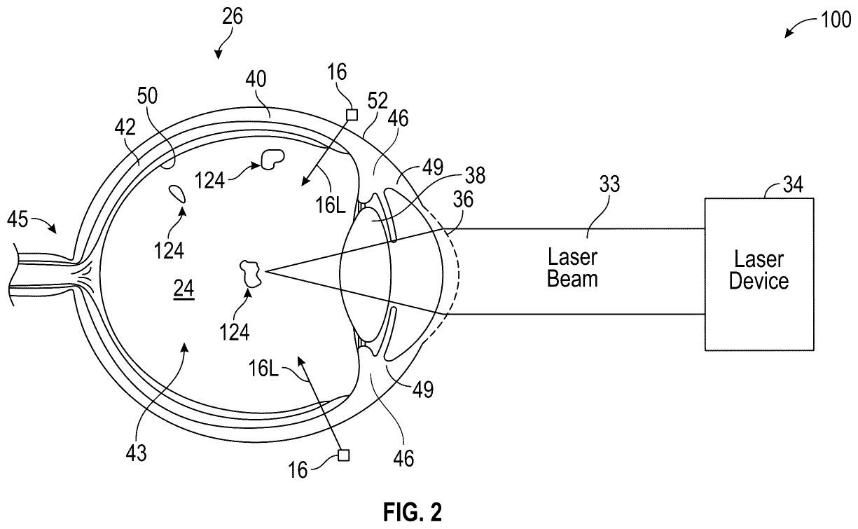

Referring to the drawings, wherein like reference numbers refer to like components, a representative visualization procedure 10 as illustrated in FIG. 1 is performed using a transscleral visualization system 12 in accordance with the present disclosure. The transscleral visualization system 12 includes an ophthalmic visualization tool 14, which is illustrated in FIG. 1 being grasped in a hand 13 of a clinician (not fully shown). The visualization tool 14 includes or more light emitters 16, a frame 18, and a processor 20 as set forth in detail below. As contemplated herein, the system 12 can be used by the clinician during the visualization procedure 10 to help view the vitreous body (“vitreous”) 24 (see FIG. 2) within an eye 26 of a patient 28.

The visualization procedure 10 could be performed in conjunction with a stereoscopic biomicroscope (“slit-lamp”) 30. As appreciated by those skilled in the art, the slit lamp 30 is typically configured to emit a light beam with clinician-controllable characteristics when viewing the interior of the eye 26. During the visualization procedure, e.g., in preparation for or concurrent with a vitreolysis or vitrectomy surgery, and with or without the slit-lamp 30, the clinician can place the visualization tool 14 directly on the eye 26. The clinician is then able to view the vitreous 24 of FIG. 2 using the uniform illumination provided by the visualization tool 14.

In one or more embodiments, the clinician may interact with the light emitter(s) 16 to selectively adjust a setting thereof. This action may occur in response to a user input signal 32, e.g., using a voice command, foot pedal activation, or use of a button, touch screen, etc. The processor 20 may receive the user input signal 32 and respond by changing one or more settings of the light emitter(s) 16, such as by adjusting a color and/or intensity setting. This may be accomplished via transmission of an electronic control signal (CC_{E}) 132 from the processor 20 to associated drive circuitry of the light emitters 16.

By way of a non-limiting example, the light emitters 16 shown schematically in FIG. 1 could include light-emitting diodes (LEDs), for instance red, green, and blue (RGB) diodes or an RGB diode array to produce and output white light. The setting(s) of the light emitters 16 could include an adjustable color setting. As the use of blue light could be advantageous when viewing the vitreous 24 of FIG. 2, the above-noted RGB LEDs could be configured to selectively emit blue light in response to the user input signal 32 in one or more implementations. Thus, the setting of the light emitters 16 may include a color of the light emitter 16, with the user input signal 32 including a requested color of the light emitter 16, for instance a surgeon's preferred color(s).

Referring now to FIG. 2, a representative laser vitreolysis surgery 100 is illustrated during which discrete pieces of the vitreous 24 move as floaters 124 within the vitreous cavity 43 of the patient's eye 26. As this occurs, the floaters 124 are individually targeted by a laser beam 33 emitted by an external laser device 34. As appreciated in the art, vitreolysis typically involves the transmission of nanosecond-length pulses of light to dissect or evaporate the floaters 124 depending on their size and location. The laser device 34 is embodied as an yttrium-aluminum-garnet (YAG) laser in a typical setup, with laser devices 34 of shorter pulse lengths being usable as part of the vitreolysis surgery 100, e.g., emerging femtosecond lasers.

In the exemplary implementation of the laser vitreolysis surgery 100, the laser beam 33 is directed into the vitreous 24 through the cornea 36 and lens 38 of the eye 26 as shown. The vitreous chamber 43 exists between the lens 38 located at the anterior of the eye 26 and the optic nerve 45 located at the posterior of the eye 26, with the vitreous 24 filling the vitreous chamber 43 in contact with the retina 50. The laser beam 33 thus passes through the vitreous 24 to fall incident upon the floaters 124, with energy imparted by the laser beam 33 ultimately reducing or evaporating the floaters 124 as understood in the art.

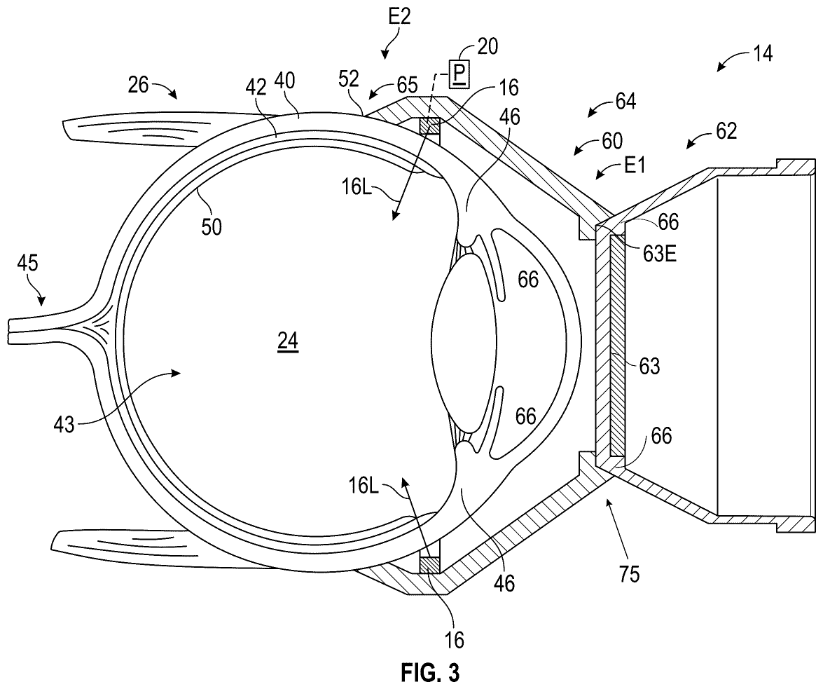

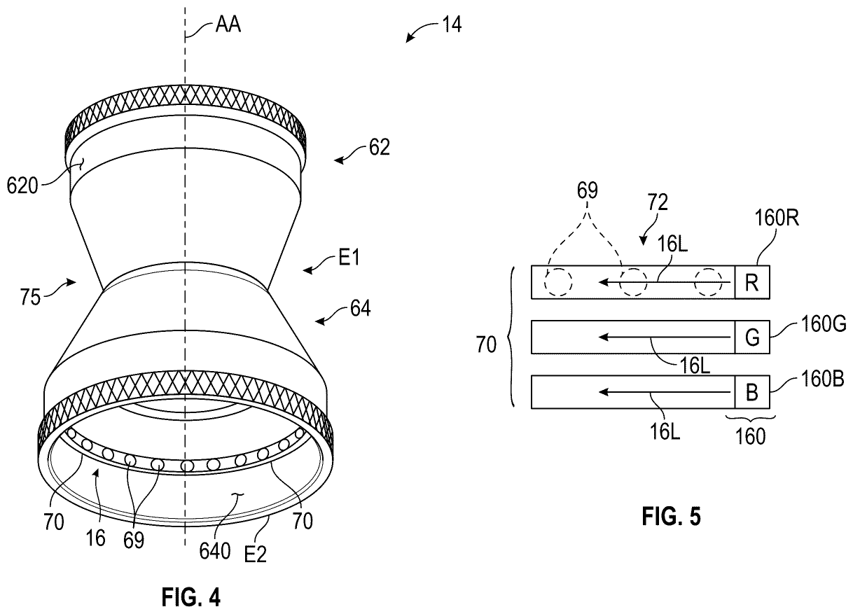

Because the floaters 124 are dynamic phase objects, the floaters 124 can be challenging to properly visualize and distinguish from the surrounding vitreous 24. To that end, the light emitters 16 are placed as shown allow emitted light 16L to pass through the sclera 40 and choroid 42 into the vitreous chamber 43. As described in detail below with particular reference to FIGS. 3 and 4, the light emitters 16 as contemplated herein are positioned proximate or in the pars plana region 46, i.e., an approximately 4-millimeter (4 mm) portion of the ciliary muscle located near the junction of the sclera 40 and the iris 49. Positioning of the light emitters 16 in this manner has various attendant benefits, including but not limited to reducing or eliminating instances of scattered light, e.g., from the lens 38 when the lens 38 is constructed as an artificial intraocular lens. The specific location of the light emitters 16 proximate the pars plana region 46 is thus intended to facilitate visualization of the vitreous 24.

Further with respect to positioning of the light emitters 16, the eye 26 when viewed from the side as in FIG. 2 has a shape formed primarily from the surrounding sclera 40. The sclera 40 thus forms the outer wall of the eye 26. Located on the sclera 40 and undersides of the eyelids (not shown) is the conjunctiva 52, i.e., a transparent mucous membrane that protects and lubricates the eye 26. The light emitters 16 are connected to a frame 60 as shown in FIG. 3, with portions of the frame 60 resting on the sclera 40 with the conjunctiva 52 positioned therebetween. The scleral-conjunctival interface may be treated in some implementations with a sticky thixotropic material to increase friction for the purpose of maintaining the desired optimal position of the light emitters 16 and reducing saccadic velocity.

Referring to FIG. 3, the ophthalmic visualization tool 14 is shown positioned on the patient's eye 26. The visualization tool 14 in this exemplary configuration includes the frame 60 and the light emitter 16, with the latter depicted as being in wired or wireless communication with the processor (P) 20. The frame 60 in this embodiment includes an annular body 62 configured to support an optical lens 63 therein, e.g., a high-diopter or wide-angle lens through which the clinician may view the vitreous 24, such as an exchangeable lens for viewing the anterior, middle, or posterior vitreous as needed during the particular visualization task. For instance, the annular body 62 could include a set of tabs 66 configured to engage a perimeter edge 63E of the optical lens 63.

A flange 64 is connected to or formed integrally with the annular body 62, e.g., at a narrowed interface or neck portion 75. The neck portion 75 could be integral with the set of tabs 66 and thus configured to support the perimeter edge 63E of the optical lens 63. The flange 64 has a first end E1 and a second end E2. The first end E1 is connected to the annular body 62. The second end E2 is configured to rest on the sclera 40 of the patient's eye 26. In this embodiment, the light emitter 16 is connected to the flange 64 proximate the second end E2 such that the light emitter 16 is configured to direct light 16L into the vitreous cavity 43. This occurs through the intervening tissue of the sclera 40 and choroid 42, and thus is trans-scleral.

At the specific location proximate the pars plana region 46, the emitted light 16L uniformly illuminates the vitreous 24 without undesirable scattering or reflection. The clinician is thus better able to track the floaters 124 as they move within the vitreous 24. The second end E2 rests on the sclera 40. Therefore, the flange 64, or at least the distal end surface 65 thereof, should be constructed of materials that would be comfortable when temporarily in contact with the eye 26. Exemplary materials include synthetic polymers such as polymer polymethyl methacrylate (PMMA), rubberized or coated plastics, silicone rubber, etc.

Referring now to FIG. 4, the flange 64 in the illustrated construction may have a frustoconical shape as shown, such that the flange 64 forms a skirt or apron that angles outward along an optical axis AA toward the eye 26. The light emitter 16 is connected to the flange 64 in proximity to the second end E2, with an inner circumferential wall 640 extending between the second end E2 and the light emitter 16. Just aft of the annular arrangement 70, i.e., between the eye 26 (FIGS. 1-3) and the first end E1, the optical lens 63 could be connected to and surrounded by the frame 60, in this instance the flange 64 thereof. Attached to the first end E1, the annular body 62 extends away from the flange 64 and provides sufficient surface area for the clinician to grasp. A collar 620 may be disposed on/connected to the annular body 62 as shown to facilitate handling and positioning in this manner.

As illustrated in FIG. 5, in one or more embodiments the light emitter 16 may include a plurality of light-emitting diodes (LEDs) 160, e.g., red (R), green (G), and blue (B) LEDs 160R, 160G, and 160B, respectively. The LEDs 160 could be connected to or arranged within an annular arrangement 70, which in turn may include one or more optical fibers 72 arranged therein. For example, a bundled plurality (“fiber bundle”) of two, three, or four of the optical fibers 72 could be used in various embodiments to connect to respective ones of the LEDs 160 and provide the desired uniform illumination along a corresponding length of the optical fibers 72. Light emission ports 69 may be connected to the optical fibers 72 of the annular arrangement 70 and spaced equally as shown to ensure uniform illumination around the circumference of the second end E2.

Embodiments of the present disclosure are described herein. The disclosed embodiments are merely examples, however, and thus other embodiments can take various and alternative forms. The drawings are not necessarily to scale. Some features could be exaggerated or minimized to show details of particular components. Therefore, specific structural and functional details disclosed herein are not to be interpreted as limiting, but merely as a representative basis for teaching one skilled in the art to variously employ the present disclosure.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

The detailed description and the drawings are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims.

What is claimed is:

1. An ophthalmic visualization tool for visualizing a vitreous body within a patient's eye, the eye having a pars plana region, the ophthalmic visualization tool comprising:
a frame having:
an annular body configured to support an optical lens; and
a flange connected to the annular body and having a first end and a second end, wherein the first end is connected to the frame and the second end is configured to rest on a scleral surface of the eye proximate the pars plana region; and
a light emitter connected to the flange proximate the second end, the light emitter being configured to direct light through the scleral surface and into a vitreous cavity of the eye, at the pars plana region, to thereby uniformly illuminate the vitreous body.

2. The ophthalmic visualization tool of claim 1, wherein the annular body includes a set of tabs configured to engage a perimeter edge of the optical lens.

3. The ophthalmic visualization tool of claim 2, further comprising:
the optical lens.

4. The ophthalmic visualization tool of claim 1, wherein the flange has a frustoconical shape, the flange having the first end and the second end.

5. The ophthalmic visualization tool of claim 1, wherein the first end of the flange is connected to the annular body and the second end is configured to rest on the scleral surface.

6. The ophthalmic visualization tool of claim 1, wherein the light emitter includes a plurality of light-emitting diodes (LEDs).

7. The ophthalmic visualization tool of claim 6, wherein the LEDs include one or more blue LEDs.

8. The ophthalmic visualization tool of claim 6, wherein at least one of the LEDs is connected to an optical fiber.

9. The ophthalmic visualization tool of claim 8, wherein the optical fiber includes a plurality of optical fibers, and wherein the LEDs are connected to the optical fibers in an annular arrangement.

10. A transscleral illumination system for visualizing a vitreous body within a patient's eye, the eye having a pars plana region, the transscleral illumination system comprising:
an ophthalmic visualization tool having:
a frame configured to support an optical lens; and
a flange having a first end and a second end, wherein the first end is connected to the frame and the second end is configured to rest on a scleral surface of the eye proximate the pars plana region; and
a light emitter connected to the flange proximate the second end, the light emitter being configured to direct light through the scleral surface and into a vitreous cavity of the eye, at the pars plana region, to thereby uniformly illuminate the vitreous body; and
a processor in communication with the light emitter, wherein a setting of the light emitter is controllable via the processor.

11. The transscleral illumination system of claim 10, wherein the frame includes an annular body having a neck portion, and wherein the neck portion is configured to support a perimeter edge of the optical lens.

12. The transscleral illumination system of claim 11, wherein the flange has a frustoconical shape inclusive of the first end and the second end, and wherein the first end is connected to the neck portion and the second end is configured to rest on the sclera at the pars plana region.

13. The transscleral illumination system of claim 10, wherein the light emitter includes a plurality of light-emitting diodes (LEDs).

14. The transscleral illumination system of claim 13, wherein the LEDs are configured in an annular arrangement.

15. The transscleral illumination system of claim 14, wherein the annular arrangement includes one or more optical fibers connected to the LEDs.

16. The transscleral illumination system of claim 10, wherein the processor is configured to adjust a setting of the light emitter in response to a user input signal.

17. The transscleral illumination system of claim 16, wherein the setting of the light emitter includes a color of the light emitter, and wherein the user input signal is a requested color of the light emitter.

18. The transscleral illumination system of claim 10, further comprising the optical lens, wherein the optical lens is configured as an exchangeable lens configured for viewing an anterior, middle, or posterior portion of the vitreous body.

19. An ophthalmic visualization tool for visualizing a vitreous body within a patient's eye, the eye having a pars plana region, the ophthalmic visualization tool comprising:
an optical lens configured for viewing an anterior, middle, or posterior portion of the vitreous body;
a frame having an annular body and a neck portion, wherein the neck portion is configured to support a perimeter edge of the optical lens;
a flange having a frustoconical shape inclusive of a first end and a second end, wherein the first end is connected to the neck portion and the second end is configured to rest on a scleral surface of the eye at the pars plana region; and
a plurality of red, green, and blue (RGB) light-emitting diodes (LEDs) configured in an annular arrangement that is connected to the flange proximate the second end, the annular arrangement being configured to direct light into a vitreous cavity of the eye through the scleral surface, at the pars plana region, to uniformly illuminate the vitreous body.

20. The ophthalmic visualization tool of claim 19, wherein the plurality of RGB LEDs have an adjustable color setting inclusive of blue light, and wherein the RGB LEDs are configured to selectively emit blue light in response to a user input signal.
